(19) United States
(12) Patent Application Publication
(10) Pub. No.: US 2006/0271097 A1
(43) Pub. Date: Nov. 30, 2006

(54) Electrolytically detachable implantable devices

(76) Inventors: Kamal Ramzipoor, Fremont, CA (US); Scott A. McGill, San Ramon, CA (US); Michael P. Wallace, Pleasanton, CA (US); Sarah Young, Fremont, CA (US); Russell B. Ford, Watsonville, CA (US)

Correspondence Address: ROBINS & PASTERNAK, 1731 EMBARCADERO ROAD, SUITE 230, PALO ALTO, CA, 94303, US

(21) Appl. No.: 11/140,690

(22) Filed: May 31, 2005

(51) Int. Cl. A61M 29/00

(52) U.S. Cl. 606/200

(57) ABSTRACT

Described herein are electrolytically detachable implantable devices and assemblies. In particular, implantable devices and assemblies that are flexible in or near the electrolytically erodable junction region are provided. Also provided are methods of using the devices and assemblies.

### FIELD OF THE INVENTION

This invention generally relates to implantable devices (e.g., embolic coils, stents, filters and other medical devices) having flexible electrolytic detachment mechanisms. In particular, disclosed herein are devices including structures that move freely at or near the electrolytic detachment junction.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids. See, e.g., U.S. Pat. No. 6,299,627. Vaso-occlusive coils having little or no inherent secondary shape have also been described. For instance, co-owned U.S. Pat. Nos. 5,690,666; 5,826,587; and 6,458,119 by Berenstein et al., describes coils having little or no shape after introduction into the vascular space. U.S. Pat. No. 5,382,259 describes non-expanding braids covering a primary coil structure.

U.S. Pat. Nos. 6,620,152; 6,425,893; 5,976,131 5,354,295; and 5,122,136, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint.

However, there remains a need for assemblies in which the implantable device can articulate with respect to the deployment mechanism. There also remains a need for assemblies in which such flexible junctions allow for efficient separation and placement of the implantable device from catheter based delivery systems.

### SUMMARY OF THE INVENTION

Thus, this invention includes implantable devices comprising novel detachment junction members as well as methods of using and making these devices. In particular, the flexible, articulating connection of the implantable device to the delivery system reduces kickback forces in the event of catheter kickback (as the coil reorients itself) during deployment as well as during detachment.

In certain aspects, the invention includes an implantable assembly comprising: a first implantable device having a proximal end and a distal end, the first implantable device comprising a loop on the proximal end; and a second loop that interlocks with the loop on the proximal end of the first implantable device, wherein the second loop comprises a metal. The first and/or second loops can be electrically insulated.

In certain embodiments, the second loop is formed from the distal end of an electrically insulated pusher wire having proximal and distal ends, wherein the electrical insulation is removed from at least a portion of the second loop to form an electrolytically erodable region on the second loop. In other embodiments, the second loop is on the distal end of a second implantable device.

In any of the assemblies described herein, the first and/or second implantable device can comprise a vaso-occlusive device, for example, a vaso-occlusive coil or a tubular structure (e.g., braid). The first and/or second implantable device (e.g., coil) may comprise one or more metals (e.g., platinum, palladium, rhodium, gold, tungsten, stainless steel, and alloys thereof such as a super-elastic metal alloy) and/or one or more polymers (e.g., biodegradable or water-soluble polymers). In certain embodiments, the polymer(s) is(are) coated onto a metal, for example to electrically insulate the implantable device(s).

Further, any of the assemblies described herein may further comprise a tensioning member, for example, a tensioning member having first and second ends, the first end attached either to the electrically insulated pusher wire.

In embodiments in which the second loop is on the distal end of a second implantable device, the assembly may further comprise a pusher wire, for example a pusher wire that is attached to the second implantable device. Any of these assemblies may further comprise one or more electrolytically erodable detachment junctions, which may be positioned anywhere on the device. In certain embodiments, the electrolytically erodable detachment junction is positioned proximal to the second implantable device. In other embodiments, the detachment junction is distal to the second implantable device and in still other embodiments, the detachment junction is internal of the second implantable device.

In another aspect, any of the assemblies described herein may further comprise a noble metal (e.g., gold or platinum) distal to the electrolytically erodable detachment junction.

In another aspect, the invention includes an implantable assembly as described herein, comprising a second implantable device, which second implantable device comprises a helically wound vaso-occlusive coil having a straight portion that extends through at least part of the lumen created by the helically wound portion. In certain embodiments, at least one of the helical winds of the coil touches the straight portion. In other embodiments, the assemblies further comprise an additional helically wound coil that touches the straight portion and extends through at least portion of the lumen of the second least one of the helical winds of the second implantable device.

In yet another aspect, the invention includes an implantable assembly as described wherein the second loop further comprises a straight portion extending proximally from the second loop and a helically wound coil wound around the straight portion.

In a still further aspect, the invention includes a method of occluding a body cavity comprising introducing any of the devices described herein into a body cavity (e.g., an aneurysm).

These and other embodiments of the subject invention will readily occur to those of skill in the art in light of the disclosure herein.

### BRIEF DESCRIPTION OF THE FIGURES

FIG. 1 is a side view, partial cross section view of an exemplary implantable device comprising a loop on its proximal end.

FIG. 2 is a side view, partial cross section view of the exemplary implantable device as shown in FIG. 1 rotated approximately 90°.

FIG. 3 is a side view of an exemplary coil assembly attached to the loop on the proximal end of the implantable device shown in FIGS. 1 and 2. The exemplary coil assembly is shown prior to attachment. Attachment may be achieved by forming a loop from the straight distal region of the exemplary coil assembly through the loop on the proximal end of the implantable device, extending the remaining straight portion back through at least a portion of the lumen of the coil assembly and attaching this end to a pusher wire.

FIG. 4, panels A to E, are side views depicting exemplary assemblies that include a conductive coil immediately distal to the detachment junction. FIG. 4A depicts an embodiment in which second loop is formed by looping the pusher wire back on itself and placing the conductive coil where the ends of the loop meet. FIG. 4B depicts a variation in which the second loop is attached to the pusher wire and the conductive coil extends distally into the second loop. FIG. 4C shows a variation of the design shown in FIG. 4A using a flat pusher wire to form the second loop. FIG. 4D shows a variation in which the flat pusher wire is folded back over the coil. FIG. 4E shows the same variation as shown in FIG. 4E and including electrically insulating material over the coil and the wire ends.

FIG. 5 is a side view of an exemplary implantable assembly as described herein. The interlocking loop structure allows for greatly enhanced flexibility of the main coil with respect to the pusher wire and detachment junction.

FIG. 6 is a side view of another exemplary implantable assembly having improved articulation with respect to the pusher wire and detachment junction.

FIG. 7 is a side view of another exemplary, flexible, implantable assembly as described herein.

FIG. 8 is a side view of an exemplary wire before it is formed into part of an implantable assembly.

FIG. 9 is a side view of an exemplary implantable assembly including the wire shown in FIG. 8 after it is formed into part of the assembly. The wire shown in FIG. 8 is formed into a structure including a detachment junction, a helically wound coil structure, a loop that interlocks with the loop on the proximal end of the main coil and an electrically conductive distal portion that passes back through the coil structure and is wound around itself distal to the detachment junction.

FIG. 10, panels A and B, depict an exemplary embodiment that includes a tensioning member. FIG. 10A shows the device prior to electrolytic detachment. FIG. 10B shows the device after electrolytic detachment.

FIG. 11 depicts another exemplary embodiment that includes a tensioning member and is shown prior to detachment.

### DESCRIPTION OF THE INVENTION

Implantable devices and assemblies comprising implantable devices are described. The devices described herein find use in vascular and neurovascular indications and are particularly useful in treating aneurysms, for example small-diameter, curved or otherwise difficult to access vasculature, for example aneurysms, such as cerebral aneurysms. Methods of making and using these devices also form aspects of this invention.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise.

As noted above, implantable devices may be conveniently detached from the deployment mechanism (e.g., pusher wire) by the application of electrical energy, which dissolves a suitable substrate at the selected detachment junction. However, many available electrolytically detachable implants are inflexible in or near the detachment junction. As a result of this inflexibility, the force exerted on the pusher wire by the operator can result in catheter kickback during placement or detachment (i.e., the tip of the catheter is displaced out of the aneurysm when the force exerted on the coil via the pusher wire is transmitted back to the catheter) and/or in inefficient detachment of the coil.

Thus, the implantable devices and assemblies comprising these devices comprise structural components that result in increased flexibility and articulation of the implantable device with respect to the deployment mechanism (e.g., pusher wire and/or catheter). For example, flexibility may be imparted by the geometry of components in or near the electrolytically erodable detachment zone.

In certain embodiments, the implantable devices exhibit flexibility due to an interlocking loop structure. For instance, the implantable device typically includes a loop structure on its proximal end, for example, a ring structure. The term “loop” as used herein is used to refers to a curved or doubled structure (thread, wire, etc.) that form a closed or partly open curve through which another structure can be passed or into which a hook may be hooked. Thus, the term includes ring-like structures as well as hook-like structures.

When the implantable device comprises a helically wound embolic coil as shown in the Figures, it may be preferable that the proximal loop is not made from a wind of the coil, but, instead, is made from an unwound portion of the wire wound into the coil structure. Alternatively, the loop on the proximal end of a helically wound coil may comprise a separate structure affixed to the proximal end of the helically wound coil. Furthermore, it is preferable that the plane formed by the proximal loop structure is substantially perpendicular to the plane created by the loops of the helically wound coil.

The loop on the proximal end of the first implantable device is attached to a second loop, typically by interlocking the loop structures. The second loop of the devices described herein comprises at least one metal, preferably an electrically erodable metal. The second loop may be partially or fully electrically insulated, for example by coating with an electrically insulated polymer. The second loop may be, for example, a loop formed from a deployment mechanism (e.g., pusher wire) and/or a loop formed from a second implantable device. The assemblies may also comprise additional implantable devices proximal to the second loop and distal to the electrolytically detachable junction zone.

In embodiments in which the assemblies include one or more implantable helically wound coils proximal to the second loop, it is preferable that the plane defined by the second loop structure be substantially perpendicular to the plane created by the loops of the helically wound coil.

In all these configurations, the interlocking loop structure allows for the free articulation of the first implantable device.

Depicted in the Figures are exemplary embodiments of the present invention in which the implantable device is depicted as an embolic device. It will be appreciated that the drawings are for purposes of illustration only and that other implantable devices can be used in place of embolic devices, for example, stents, filters, and the like. Furthermore, although depicted in the Figures as embolic coils, the embolic devices may be of a variety of shapes or configuration including, but not limited to, braids, wires, knits, woven structures, tubes (e.g., perforated or slotted tubes), injection-molded devices and the like. See, e.g., U.S. Pat. No. 6,533,801 and International Patent Publication WO 02/096273. It will also be appreciated that the assemblies can have various configurations as long as the required flexibility is present.

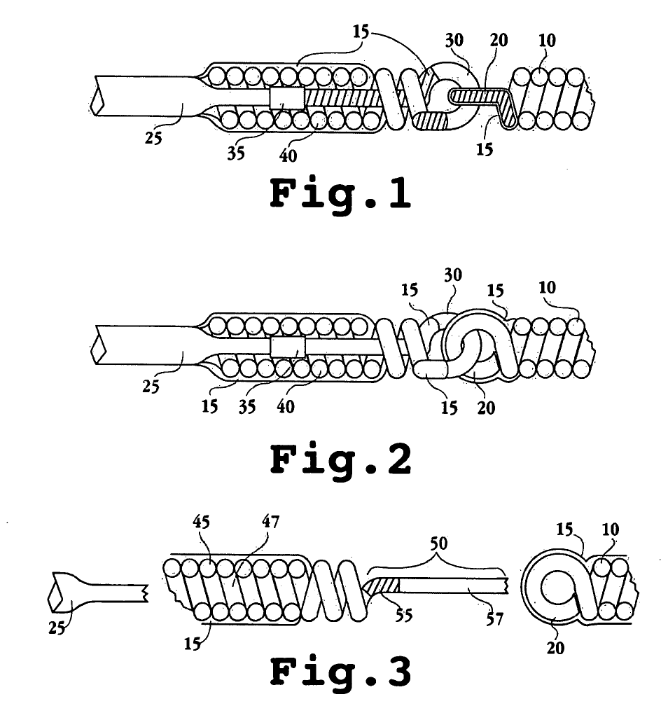

FIGS. 1 and 2 are side and cross-section views of an exemplary flexible implantable device as described herein, in which the interlocking loop structure that allows flexibility of the implantable vaso-occlusive coil also serves the electrolytically erodable detachment junction. In particular, the implantable coil 10 (also called main coil) comprises an electrically insulated 15 proximal loop 20 attached a second loop 30. The second loop 30 comprises an electrolytically detachable region.

The second loop 30 may be formed from the pusher wire 25 itself, for example by removing the electrically insulating coatings from a looped portion 30 of the pusher wire 25 and/or by coating the looped portion of the pusher wire with a noble metal such as platinum or gold to accurately bound the length of the detachment zone. A coating with noble metal may also for more efficient electrolytic detachment at the loop (FIG. 11) and/or improve the ability to detect detachment.

Alternatively, as shown in FIGS. 1 and 2, the second loop 30 may be formed from a second assembly 40, which is proximal to the loop 20 on the implantable device 10. The second assembly 40 comprising the second loop is then affixed directly or indirectly to the pusher wire 25. As noted above, the second assembly may be a coil structure (e.g., a coil made up of two or more helical winds), a tubular structure (e.g., metal and/or polymer, preferably of a substantially uniform thickness), a filter, a stent, or the like.

In the variations as shown in FIGS. 1 and 2, the main coil loop 20 is attached to the pusher wire 25 via a proximal coil assembly 40, which assembly is in turn affixed to the distal end 35 of the pusher wire 25 such that the pusher wire 25 and proximal coil assembly 40 are in electrical contact. In the embodiments depicted, the proximal assembly 40 comprises a helically wound electrical conductive core wire (e.g., stainless steel wire) surrounded by an electrically insulating coating 15 (e.g., a polymer polyimide). The electrically insulating polymer 15 is removed in the region of the second assembly 40 that forms the second loop 30.

Detachment of the implantable coil 10 from the proximal coil assembly 40 (and hence from the pusher wire 25) occurs when an electrical current is passed through the pusher wire 25 to the electrically erodable loop 30 formed by the second coil assembly 40. The proximal assembly 40 is removed when the pusher wire 25 is removed.

FIG. 3 shows an exemplary proximal coil assembly 40 prior to formation of the second loop structure 30 and attachment to the pusher wire 25. The proximal coil assembly 40 is formed by winding an electrically insulated core wire 45 into a coil like structure that includes a straight (unwound) tail portion 50 at one end. The electrically insulating coating 15 is removed from the unwound tail portion 50 by any suitable means, including but not limited to, mechanical processes such as the use of a sharp object, abrasive spray techniques, chemical processes, the use of a laser or like focused energy source. Optionally, the distal end 57 of region from which the insulation has been removed is coated with a noble metal such as platinum or gold to create a region of wire 55 and a region of wire coated with a noble metal 57. The optional noble metal coating effectively alters the impedance of the system during electrolytic detachment, allowing detection of detachment using existing electronic systems.

The tail portion 50 is then looped though the electrically insulated loop 20 on the proximal end of the implantable coil 10, inserted back though the lumen 47 (inner diameter or ID) of the wound portion 45 of the proximal coil assembly and attached to the distal end of a pusher wire 25, such that electrical conductivity between the second coil 45 and pusher wire 25 is attained. The looped back tail portion 50 can be attached to the pusher wire 25 by any suitable means, for example the use of adhesives. The second loop portion 30 forms a detachment zone so that upon application of a suitable electrical current, the second loop 30 dissolves and the main coil 10 is released into the target body cavity.

Preferably, the electrically conductive erodable portion of the loop 30 has a narrow range of circumferential contact with surrounding body fluids, so that erosion will be focused. By “focused” is meant that erosion will be limited to a narrow circumferential band, rather than a broad one; this will result in quicker erosion through the thickness of the electrically conductive portions.

The second coil 40 may be wound in a closed or open pitch. In certain embodiments (FIGS. 1 and 2), the proximal end of the second coil is wound in a closed pitch while the distal end (near to the tail that is formed into the proximal pusher wire loop of the interlocking loops) is wound in an open pitch. All or some of the second coil may be electrically insulated. For example, as shown in FIG. 1, the open pitch portion may be un-insulated which allows electrolytes to contact the non-degradable section of the uninsulated wire that extends through the center of the coil.

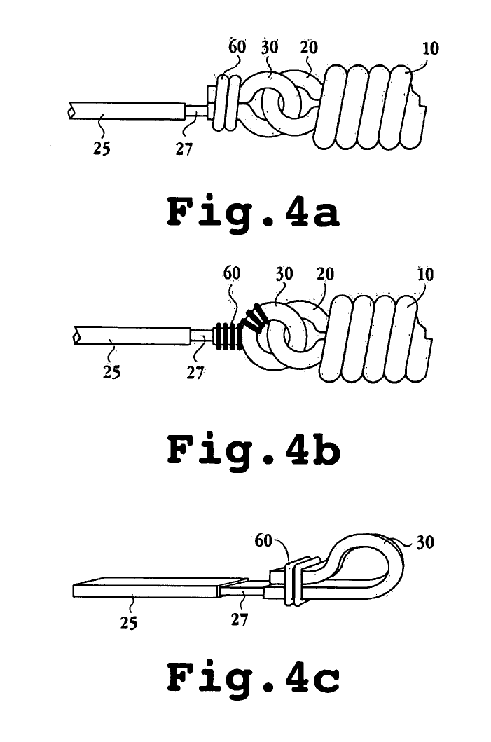

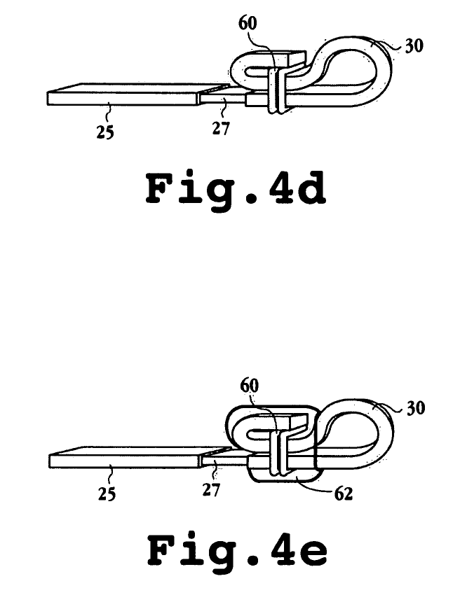

FIGS. 4A-E are partial cross-section, side views of exemplary implantable assemblies as described herein comprising a first implantable coil 10 (also called main coil) comprising a loop on its proximal end 20. The assemblies further comprise a second loop 30 that interlocks with the proximal loop 20 on the main coil 10. The interlocking ring geometry allows for freedom of movement of the main coil 10. In addition, an optional electrically conductive coil 60 is shown surrounding the second loop 30 immediately distal to an electrolytically erodable junction 27. Typically, the electrolytically erodable region 27 is created by removing electrical insulation from an electrically conductive pusher wire 25 in a region near the conductive coil 60.

The optional conductive coil 60 may be attached to the pusher wire 25 and/or second loop 60 by any suitable means, for example by welding, crimping, interference fit, or the like. It will be apparent that the conductive coil shown in FIGS. 4A-C can be any shape or construction, so long as it comprises a conductive material.

FIG. 4A shows an embodiment in which the second loop 30 is formed by looping back a portion of the insulated pusher wire 25 and securing the platinum coil 60 at the loop closure area. FIG. 4B shows a variation of this design in which the second loop 30 is attached to the pusher 25 wire and in which the platinum coil 60 extends distally to the second loop 30. FIG. 4C shows a variation of the design shown in FIG. 4A using a flat pusher wire 25 to form the second loop 30. The flat wire design allows for increased flexibility as well as allowing for a smaller diameter in the area of contact with the platinum coil 60, which may improve bond strength. The flat wire design may also improve the speed at which electrolytic detachment occurs, perhaps due to the increased surface area.

FIG. 4D shows a variation of the design of FIG. 4C in which the flat pusher wire 25 is folded back over the coil 60. This design increases the mechanical (tensile) strength of the design and reduces the need for additional elements or process (e.g., welding, use of adhesives, etc.) to hold the components together. FIG. 4E shows a variation of the design shown in FIG. 4D and includes an electrically insulating material 62 over the coil 60 and the end of the wire 25. The electrically insulating material 62 helps reduce or prevent dissolution of the wire 25 in the presence of the electrolyte and also reduces the likelihood that the coil 60 will come into electrical contact with other implantable devices, for example coils already implanted into the aneurysm. The electrically insulating material 62 may be any of the materials described below including, but not limited to, polymers such as PET, adhesives and the like.

It is to be understood that although the ring on the proximal end of the implantable device is depicted in FIGS. 4A-C as attached to the pusher wire via a second ring structure, other arrangements may be used to attach the pusher wire to the ring on the proximal end of the implantable coil. For example, proximal coil ring may be attached to the pusher wire directly or by any other suitable structures, including, but not limited to, hook structures, FIG. 8 structures and the like.

In the embodiments shown in FIGS. 4A-C, the assemblies are designed such that upon application of electrical current, the detachment junction 27 just proximal to the conductive coil 60 dissolves and the main coil 10, insulated rings 20, 30 and conductive coil 60 are all implanted into the selected body cavity. However, it is to be understood that the present invention also encompasses assemblies in which detachment occurs closer to the insulated ring 30 on the proximal end of the implantable device, so long as the geometry of components in or near the detachment junction allow the implantable device to move freely. More than one detachment zone may also be included in the assemblies.

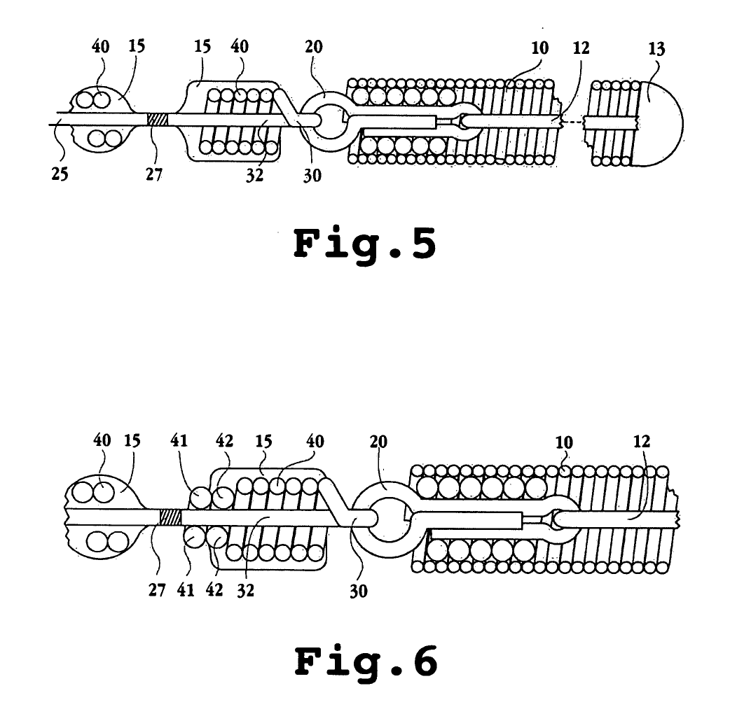

FIG. 5 shows an exemplary implantable assembly of the invention comprising a main coil 10 having a first loop 20 on its proximal end. In this embodiment, the first loop 20 is attached to the main coil 10 via a suture or wire 12 that extends through the lumen of the main coil 10 to a cap 13 on the distal end of the main coil 10. The attachment to the suture/wire 12 may be by any suitable mechanism, including welding, tying, melting, or by looping as shown in FIG. 5. The first loop 20 may also be attached to one of winds of the main coil 10, for example by spot welding.

The first loop 10 interlocks with a second loop 30 extending from the distal end of a second coil 40. In the embodiment depicted in FIG. 5, the second loop 30 is created from an unwound portion of a second helically wound coil 40, essentially as described above with regard to FIG. 3. However, it will be apparent that the second loop 30 may also be a separate structure attached to the second coil 40 by any suitable means.

The unwound portion of the second coil used for form the second loop 30 preferably includes a sufficient amount of unwound material to form the second loop 30 and to include a straight portion 32 that can be extended back through the lumen of the second coil 40, where it can be attached to the pusher wire 25 by any suitable means. Pusher wire 25 comprises an electrically conductive material, for example, stainless steel and is preferably electrically insulated, except in region(s) where the electrical insulation is removed to form an electrolytically erodable joint.

The second coil 40 is typically electrically insulated, for example by coating a metal coil (e.g., stainless steel) with an electrically insulating material such as a polymer. The electrically insulating material 15 can be removed from the pusher wire 25 and /or from a portion of the looped back portion of the second coil 40 to form a detachment zone 27. As will be apparent, the detachment zone can be proximal, distal or interior to, the second coil 40.

In the embodiments depicts in FIGS. 5-9, the detachment zone 27 is internal to the second coil 40, for example in an electrolytically erodable area created when the unwound portion of the second coil 32 is extended back through the lumen of the second coil 40 and the electrical insulation removed in order to create the detachment joint 27. Alternatively, the detachment zone 27 may be created by extending a suitably designed pusher wire 25 through the lumen of the second coil assembly 40. A coating of a noble metal such as platinum or gold may be added distal to the detachment zone 27 to enhance the impedance of the system and allow for detection of the detachment signal.

FIG. 6 shows another exemplary embodiment similar to that shown in FIG. 5, including main coil 10 with proximal loop 20, loop attachment mechanism 12 through the lumen of the main coil 10, electrically insulated second coil 40 proximal to main coil 10, and an unwound portion that forms the second loop 30 and a straight portion 32 that is looped back through the lumen of the second coil 40. In this variation, an uninsulated portion 41, 42 of the electrically conductive second coil 40 is contacted with looped back portion of the assembly (or pusher wire) distal to the detachment zone 27.

The electrically conductive coil winds 41, 42 may be contacted with the looped back portion 32 (or pusher wire 25) in any suitable way, for example by welding and/or crimping of the coil winds 41, 42, so long as the coil and looped back portion 32 of the second coil 40 (or pusher wire 25) including the detachment zone 27 are in electrical contact.

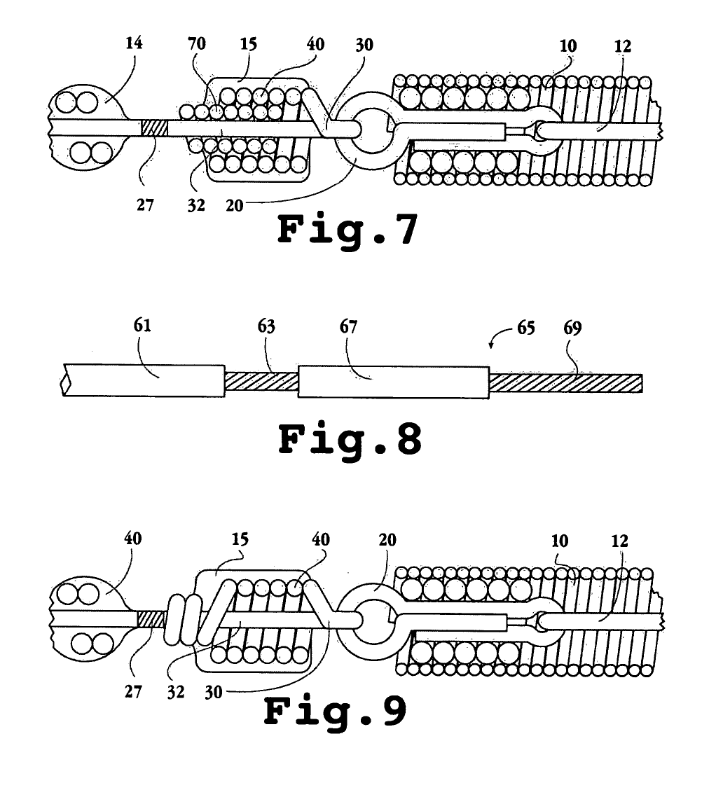

FIG. 7 shows yet another variation in which the coil assembly 40 proximal to the main coil 10 comprises a second electrically insulated coil 40 which second coil 40 at least partially surrounds a third electrically conductive coil 70. The third electrically conductive coil 70 is in electrical contact with the looped back portion 32 (or pusher wire 25) distal to the detachment zone 27 and extends at least partially through the lumen of the second coil 40.

FIG. 8 shows an exemplary wire 65 that may be wound to form the second coil assembly 40 and second loop 30 as shown in FIG. 9. As shown in FIG. 8, prior to winding, the electrically conductive wire 65 (e.g., stainless steel, platinum or gold) comprises, in a proximal to distal direction, a first electrically insulated region 61, a region in which the electrically insulating coating has been removed 63, a second electrically insulated region 67 and a second region in which the electrically insulating coating 69 has been removed and, if the wire is stainless steel, optionally coated with a noble metal such as platinum or gold. It will be apparent that if the wire 65 comprises platinum or gold, the electrically insulating coating on the distal-most region 69 may be omitted.

The wire of FIG. 8 is then formed into the second coil assembly 40 shown in FIG. 9 as follows. The first electrically insulated region 61 is wound into a helically shaped coil 40. The first electrically uninsulated region 63 remains unwound to form the detachment junction 27 shown in FIG. 9. The second electrically insulated region 67 is wound into a helically shaped coil 40, formed into the second loop 30 and extended back through the lumen of the second coil 32. The distal most region 69 from which the electrically insulation has been removed and the wire optionally coated with a noble metal is wound around the looped back portion 32 just distal to the detachment junction 27.

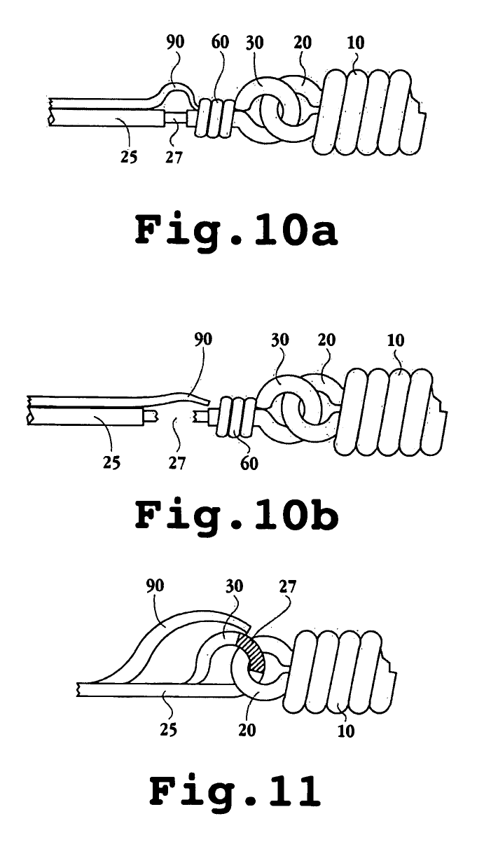

The devices and assemblies described herein may further comprise additional elements and members. For example, as shown in FIGS. 10A-B and 11, the device or assembly may further comprise a tensioning member. The tension member facilitates detachment, for example by exerting pressure on the implantable assembly (FIGS. 10A and 10B) or by keeping regions adjacent to the attachment zone from interfering with separation of the implantable device from the pusher wire (FIG. 11).

FIGS. 10A and 10B show an embodiment in which the implantable device comprises a tensioning member 90 that exerts a force on the implantable coil assembly. FIG. 10A shows the assembly prior to electrolytically induced detachment, including main coil 10, first loop 20 which interlocks with second loop 30, conductive coil 60 distal to detachment junction 27. Tensioning member 90 applies force to the main coil 10 distal to the detachment zone 27 that aids in separating the coil assembly from the pusher wire 25 after electrolytic dissolution of the detachment joint 27. As shown in FIG. 10B, upon dissolution of the electrolytically erodable detachment zone 27, the force exerted by the tensioning member 90 on the coil assembly helps to ensure complete separation of the implantable assembly from the pusher wire 25. The tensioning member 90 and pusher wire 25 can then be readily removed from the subject. The tensioning member 90 can be, for example a compressible material such as a spring.

FIG. 11 shows an embodiment in which an electrically conductive insulated pusher wire 25 is formed into the second loop 30. A portion of the insulation is removed from the region of the pusher wire 25 forming the second loop 30 in order to create an electrolytically erodable region 27 positioned on the second loop 30. Tensioning member 90 is attached to as shown such that upon electrolytically-induced dissolution of the detachment zone 27, the undissolved portions of the second loop 30 are inhibited from falling into the insulated loop 20 attached to the main coil 10, and, accordingly, the main coil 10 will separate readily from the looped pusher wire 25.

With regard to particular materials used in the implantable devices and assemblies of the invention, it is to be understood that the implantable devices or assemblies may be made of a variety of materials, including but not limited to metals, polymers and combinations thereof, including but not limited to, stainless steel, platinum, kevlar, PET, carbothane, cyanoacrylate, epoxy, poly(ethyleneterephthalate) (PET), polytetrafluoroethylene (Teflon™), polypropylene, polyimide polyethylene, polyglycolic acid, polylactic acid, nylon, polyester, fluoropolymer, and copolymers or combinations thereof. See, e.g., U.S. Pat. No. 6,585,754 and 6,280,457 for a description of various polymers. Different components of the devices and assemblies may be made of different materials.

In embodiments in which the implantable device comprises an embolic coil, the main coil may be a coiled and/or braided structure comprising one or more metals or metal alloys, for example, Platinum Group metals, especially platinum, rhodium, palladium, rhenium, as well as tungsten, gold, silver, tantalum, stainless steel and alloys of these metals. Preferably, the comprises a material that maintains its shape despite being subjected to high stress, for example, “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” The main coil may also comprise a shape memory polymer such as those described in International Publication WO 03/51444. The implantable device is preferably electrically insulated, for example, by coating a metallic coil (e.g., stainless steel, platinum) with one or more electrically insulating materials, for example one or more polymers such as polyimide.

The implantable device may also change shape upon release from the deployment mechanism (e.g., pusher wire), for example change from a linear form to a relaxed, three-dimensional configuration upon deployment.

Pusher wire 25 typically comprises an electrically conductive material such as stainless steel, platinum, gold, etc. The pusher wire or other elements may be made of, or coated with, a material such as polytetrafluoroethylene (e.g., Teflon™) and desirably extends all the way to the proximal end of the catheter. The pusher wire 25 may be rotatable and axially moveable with respect to the device. Pusher wire can also act as a guidewire and may be used to provide a pathway through tortuous vasculature for the device to follow.

As noted above, the materials used for the various electrically insulating members and layers discussed herein may be flexible polymeric coatings or layers such as polyfluorocarbons, polyurethane, polyethylene, polypropylene, polyimides, silicone polymers, or other suitable polymeric materials. In a preferred embodiment, the coating comprises parylene, which is readily deposited on a substrate in uniform layer, for example by vacuum deposition. Such polymeric materials are generally flexible, have good electrical insulation properties, and are amenable to removal, for example to create an electrolytically erodable zone at a selected position on the assembly. The same electrically insulating materials, such as polymers, may be used in various elements of the devices and assemblies described herein. Alternatively, different materials may be used in different elements. For example, it may be preferable to use a biodegradable or water-soluble polymer to electrically insulate the implantable device while using a different polymer on the elements that are not implanted (e.g., pusher wire, or, in certain embodiments, the proximal assembly that comprises the second loop). In certain embodiments, the preferred electrically insulating material used for the proximal assembly is polyimide.

The electrically insulating coating(s) can be deposited on the device using any suitable technique, including, but not limited to spray or vacuum deposition, dip coating, use of adhesives, heating to melt, heat shrink techniques and the like.

The devices described herein may also comprise additional components, such as co-solvents, plasticizers, coalescing solvents, bioactive agents, antimicrobial agents, antithrombogenic agents (e.g., heparin), antibiotics, pigments, radiopacifiers and/or ion conductors which may be coated using any suitable method or may be incorporated into the element(s) during production. See, e.g., U.S. Pat. No. 6,585,754 and WO 02/051460, U.S. Pat. No. 6,280,457. The additional components can be coated onto the device and/or can be placed in the vessel prior to, concurrently or after placement of one or more devices as described herein.

One of more of the elements may also be secured to each other at one or more locations. For example, to the extent that various elements are thermoplastic, they may be melted or fused to other elements of the devices. Alternatively, they may be glued or otherwise fastened. Furthermore, the various elements may be secured to each other in one or more locations.

Methods of Use

The implantable devices described herein are often introduced into a selected site using the procedure outlined below. This procedure may be used in treating a variety of maladies. For instance in the treatment of an aneurysm, the aneurysm itself will be filled (partially or fully) with the vaso-occlusive devices as described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the vaso-occlusive device at the distal end, is advanced through the catheter.

Once the selected site has been reached, the vaso-occlusive device is extruded using the pusher wire such that the electrolytically cleavable junction (e.g., a GDC-type junction that can be severed by application of heat, electrolysis, electrodynamic activation or other means) as described above. Additionally, the vaso-occlusive device can be designed to include multiple detachment points, as described in co-owned U.S. Pat. No. 6,623,493 and 6,533,801 and International Patent publication WO 02/45596. They are held in place by gravity, shape, size, volume, magnetic field or combinations thereof.

It will also be apparent that the flexibility imparted by the geometry of the devices described herein allows the operator can remove or reposition (distally or proximally) the implantable device. For instance, the operator may choose to insert a device as described herein, before detachment, move the pusher wire to place the device in the desired location.

Modifications of the procedure and devices and assemblies described above, and the methods of using them in keeping with this invention will be apparent to those having skill in this mechanical and surgical art. These variations are intended to be within the scope of the claims that follow.

What is claimed is:

1. An implantable assembly comprising:
a first implantable device having a proximal end and a distal end, the first implantable device comprising a loop on the proximal end; and
a second loop that interlocks with the loop on the proximal end of the first implantable device, wherein the second loop comprises a metal.

2. The implantable assembly of claim 1, wherein the second loop is formed from the distal end of an electrically insulated pusher wire having proximal and distal ends, wherein the electrical insulation is removed from at least a portion of the second loop to form an electrolytically erodable region on the second loop.

3. The implantable assembly of claim 1, wherein the second loop is on the distal end of a second implantable device.

4. The implantable assembly of claim 1, wherein the first implantable device comprises a vaso-occlusive device.

5. The implantable assembly of claim 4, wherein the vaso-occlusive device comprises a coil and the coil comprises a metal.

6. The implantable assembly of claim 5, wherein the metal is selected from the group consisting of platinum, palladium, rhodium, gold, tungsten and alloys thereof.

7. The implantable assembly of claim 5, wherein the metal is stainless steel or super-elastic metal alloy.

8. The implantable assembly of claim 3, wherein the vaso-occlusive device comprises a tubular braid.

9. The implantable assembly of claim 1, wherein the first implantable device further comprises a polymer coating.

10. The implantable assembly of claim 9, wherein the polymer is biodegradable material.

11. The implantable assembly of claim 2, further comprising a tensioning member having first and second ends, the first end attached to the electrically insulated pusher wire.

12. The implantable assembly of claim 1, wherein the loop on the proximal end of the first implantable device is electrically insulated.

13. The implantable assembly of claim 12, wherein the second loop is electrically insulated.

14. The implantable assembly of claim 3, wherein the second implantable device comprises a helically wound vaso-occlusive coil.

15. The implantable assembly of claim 3, wherein the second implantable device is electrically insulated.

16. The implantable assembly of claim 3, further comprising a pusher wire.

17. The implantable assembly of claim 16, wherein the pusher wire is attached to the second implantable device.

18. The implantable assembly of claim 3, further comprising
an electrolytically erodable detachment junction proximal to the second implantable device.

19. The implantable assembly of claim 18, further comprising a noble metal distal to the electrolytically erodable detachment junction.

20. The implantable assembly of claim 19, wherein the noble metal is gold or platinum.

21. The implantable assembly of claim 14, wherein the helically wound vaso-occlusive coil further comprises a straight portion that extends through at least part of the lumen created by the helically wound portion.

22. The implantable assembly of claim 21, wherein at least one of the helical winds of the coil touches the straight portion.

23. The implantable assembly of claim 21, further comprising an additional helically wound coil that touches the straight portion and extends through at least portion of the lumen of the second least one of the helical winds of the second implantable device.

24. The implantable assembly of claim 3, wherein the second loop further comprises
a straight portion extending proximally from the second loop and
a helically wound coil wound around the straight portion.

25. The implantable assembly of claim 16, further comprising a tensioning member having first and second ends, the first end attached to the pusher wire and further wherein the pusher wire is electrically insulated.

26. A method of occluding a body cavity comprising introducing an implantable assembly according to claim 1 into the body cavity.

27. The method of claim 26, wherein the body cavity is an aneurysm.
